(19) United States
(12) Patent Application Publication
(10) Pub. No.: US 2025/0354632 A1
(43) Pub. Date: Nov. 20, 2025

(54) TEARABLE HEAT SHRINK TUBING

(71) Applicant: Zeus Company LLC, Orangeburg, SC (US)

(72) Inventors: Tyler Poole, Lexington, SC (US); John Richard Campanelli, West Columbia, SC (US); Josh Fogle, Columbia, SC (US)

(21) Appl. No.: 19/211,916

(22) Filed: May 19, 2025

Related U.S. Application Data
(60) Provisional application No. 63/649,768, filed on May 20, 2024.

(51) Int. Cl. F16L 11/12

(52) U.S. Cl. CPC F16L 11/12

(57) ABSTRACT

The present disclosure provides heat shrink tubings prepared from one or more one poly(ether-block-amide) (PEBA) resins. Certain heat shrink tubings have a recovery ratio (RR) greater than about 1.05:1 and/or are at least partially tearable.

### CROSS-REFERENCE TO RELATED APPLICATIONS

The present application claims the benefit of U.S. Provisional Patent Application No. 63/649,768, filed May 20, 2024, which is incorporated herein by reference in its entirety.

### FIELD OF THE INVENTION

The present application is directed to heat shrink polymeric tubing and methods for making such heat shrink polymeric tubing included as a component of a catheter assembly.

### BACKGROUND OF THE INVENTION

Tubing extruded from medical grade polymers are key components in many state-of-the-art medical devices. Devices that find use of medical grade tubing include patient monitoring equipment, imaging devices, in vitro diagnostic devices, implantable devices, and interventional devices. Medical device manufacturers utilize tubing components comprising a wide variety of polymeric materials and formulations to achieve desired functionality for specific applications. The specific formulation selected for a medical tubing component is heavily dependent upon the application in which the tubing will be used. When materials of construction are selected for a specific medical device/application there are several criteria that must be considered. Some of these criteria include cost, regulatory compliance, biocompatibility, means of sterilization, and performance. Material selection and formulation is key to ensure a tubing component will meet the needs of a medical device manufacturer.

### SUMMARY OF THE INVENTION

The present disclosure relates to heat shrink tubing that exhibits tearability in the longitudinal direction manufactured using non-fluorinated polymeric resin blends. Tearability may refer to the ability of a tubing to be torn apart into substantially equal halves along the longitudinal axis of the tubing after a cut is initiated at one end of the tubing. The cut at one end of the length of tubing serves as “tear tabs” that are grasped by hand and used to tear the tubing apart down its length. A tear force can be applied by grasping or clamping both tear tabs and applying force to both tear tabs in opposite directions directed away from the central axis of the tubing. When a tear force is applied in this way, the length of tearable tubing can be torn apart into substantially equal halves.

It is to be understood that the tearable tubing of the present invention has a substantially uniform wall thickness, e.g., the tubing contains an annular cross-section and is not manufactured such that there are thin or scored wall sections to promote the propagation of the tear down the length. It is also to be understood that the compositions of tearable tubings disclosed herein are largely uniform along the length and cross-section of the tubing (e.g., material composition is homogeneous throughout the tubing article)

Various embodiments provide an extruded tube comprising a blend of non-fluorinated polymeric resins that can be torn longitudinally after a cut is initiated at one end of the tube. The formulation of more than one non-fluorinated polymeric resins is such that the final tube exhibits tearability (e.g., continuous crack propagation) in the longitudinal direction when torn after a cut is initiated at one end of the tube.

Various embodiments provide an extruded tube comprising a blend of non-crosslinked polymeric resins that can be radially expanded in a secondary process to form a tearable heat shrink tubing. Upon exposure to elevated temperatures, the expanded tube comprising the non-fluorinated polymeric resin blend will recover substantially in the radial direction (e.g., a heat shrink tubing). A small cut can be initiated at one end of the tube and by applying force in opposite directions to the tear tabs, the tube can be longitudinally torn apart into substantially equal halves either before or after the heat shrink tubing is exposed to elevated temperatures to induce radial recovery.

Various embodiments provide a tearable and heat shrinkable tube comprising a non-crosslinked polymeric resin blend such that the tube can be removed and discarded from an underlying substrate or composite structure after recovery.

Various embodiments provide an expanded tube that can be placed over a catheter shaft assembly and used as a processing aid in a catheter manufacturing process. Through careful selection of the underlying outer jacket materials with a melting temperature lower than the recovery temperature of the tearable heat shrink tubing disclosed herein, the assembly can be heated to induce radial recovery of the heat shrink tubing which will force the molten outer jacket to “reflow” and form a tight bond with underlying catheter components. After this reflow step, a small cut can be initiated at one end of the heat shrink tubing and torn away from the underlying materials to produce a finished composite catheter shaft.

Various embodiments provide a tearable and heat shrinkable tube that has been treated with radiation to enhance the recovery properties of the expanded tube when exposed to elevated temperatures. The tearable and heat shrinkable tube that has been treated with radiation is preferably not substantially crosslinked, for example, to prevent the ability of the material to flow above the melt temperature, inhibit flexibility, or bond with underlying substrates upon recovery. The initial extruded tube comprising a non-crosslinked polymeric resin blend can be subjected to a low dose of radiation (e.g., e-beam, gamma, etc.) to induce branching and partially crosslink the input tube. The input tube can then be radially expanded in a secondary process to form a tearable heat shrink tubing.

The disclosure includes, without limitation, the following embodiments.

Embodiment 1: A heat shrink tubing comprising PEBA, wherein the heat shrink tubing has a recovery ratio (RR) greater than about 1.05:1, and wherein the expanded heat shrink tubing is at least partially tearable.

Embodiment 2: The heat shrink tubing of Embodiment 1, consisting essentially of non-crosslinked PEBA.

Embodiment 3: The heat shrink tubing of Embodiment 1 or 2, comprising less than 60% by weight of crosslinked polymer.

Embodiment 4: The heat shrink tubing of any of Embodiments 1-3, comprising PEBA as the only polymer.

Embodiment 5: The heat shrink tubing of any of Embodiments 1-4, comprising a single PEBA resin.

Embodiment 6: The heat shrink tubing of any of Embodiments 1-4, comprising two or more PEBA resins.

Embodiment 7: The heat shrink tubing of any of Embodiments 1-6, comprising one or more polymers in addition to the PEBA, e.g., selected from the group consisting of polyamides, polyethers, polyesters, and copolymers, blends, or derivatives thereof.

Embodiment 8: The heat shrink tubing of any of Embodiments 1-7, further comprising one or more additives, e.g., selected from the group consisting of lubricants, colorants, fillers (e.g., conductive or radiopaque filler), stabilizer (e.g., radiation stabilizer or antioxidant), anti-tack agent, antimicrobial agent, or any combination thereof.

Embodiment 9: The heat shrink tubing of any of Embodiments 1-8, comprising no fluorinated polymer resin.

Embodiment 10: The heat shrink tubing of any of Embodiments 1-9, wherein the heat shrink tubing has a Tear Index greater than about 0.1.

Embodiment 11: The heat shrink tubing of any of Embodiments 1-10, wherein the heat shrink tubing has a Tear Index greater than about 0.5.

Embodiment 12: The heat shrink tubing of any of Embodiments 1-11, wherein the heat shrink tubing has a Tear Index greater than about 0.9.

Embodiment 13: The heat shrink tubing of any of Embodiments 1-12, wherein the heat shrink tubing has a Tear Index greater than about 0.95.

Embodiment 14: The heat shrink tubing of any of Embodiments 1-13, wherein the heat shrink tubing has a recovery ratio (RR) greater than about 1.05:1.

Embodiment 15: The heat shrink tubing of any of Embodiments 1-14, wherein the heat shrink tubing has a recovery ratio (RR) greater than about 1.3:1.

Embodiment 16: The heat shrink tubing of any of Embodiments 1-15, wherein the heat shrink tubing has a recovery ratio (RR) greater than about 1.6:1.

Embodiment 17: The heat shrink tubing of any of Embodiments 1-16, wherein the heat shrink tubing has a recovery ratio (RR) greater than about 2.0:1.

Embodiment 18: A medical device comprising the heat shrink tubing of any of Embodiments 1-17.

Embodiment 19: The medical device of Embodiment 18, wherein the medical device is a catheter.

Embodiment 20: The medical device of Embodiment 18, wherein the heat shrink tubing is in recovered form.

It will be apparent to those skilled in the art that other embodiments of the invention are possible and that the examples presented here are not intended to be exhaustive. These and other features, aspects, and advantages of the present disclosure will be apparent from a reading of the following detailed description together with the accompanying drawings, which are briefly described below. The present disclosure includes any combination of two, three, four, or more features or elements set forth in this disclosure or recited in any one or more of the claims, regardless of whether such features or elements are expressly combined or otherwise recited in a specific embodiment description or claim herein. This disclosure is intended to be read holistically such that any separable features or elements of the disclosure, in any of its aspects and embodiments, should be viewed as intended to be combinable, unless the context of the disclosure clearly dictates otherwise.

### DETAILED DESCRIPTION

The present invention will now be described more fully hereinafter with reference to certain examples, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The present disclosure provides non-fluorinated polymeric resin blends for use in the manufacture of tearable tubing and tubing produced using such resin blends. In various embodiments, the tearable tubings disclosed herein are prepared from one or more non-fluorinated polymeric resins that are essentially free of crosslinks. In some embodiments, the tearable tubings disclosed herein are prepared from more than one poly(ether-block-amide) (PEBA) resins. “Resin” as used herein may refer to a material consisting essentially of a given type of polymer (e.g., a copolymer) or two or more polymers/copolymers. Resins are typically provided in solid form (e.g., as solid pellets), although they are not limited thereto (with other forms including, but not limited to, powders, pastes, granules, dispersions, solutions, gels, and the like). In some embodiments, the tearable tubings disclosed herein may be prepared from a resin blend comprising, consisting of, or consisting essentially of non-fluorinated polymeric resins in one or more of the forms noted herein. Additionally, the non-fluorinated polymeric resin blends used to prepare the tearable tubings of the present invention may comprise, consist of, or consist essentially of more than one PEBA resin in one or more of the forms noted herein. In some cases, a “resin” as used herein may contain one or more additional components as additives and/or one or more additional components can be added thereto (e.g., such as a lubricant, colorant, filler, and the like). In other embodiments, one or more additional components (in granular, powder, or pellet form or in the form of a gel or liquid) can be included with the non-fluorinated polymeric resin blend (e.g., a PEBA resin blend) and extruded therewith. As such, the tearable tube ultimately produced can comprise, in some embodiments, one or more such additional component(s).

In some embodiments, the disclosed non-crosslinked tearable tubes comprise, consist essentially of, or consist of one or more polymers such as polyamides, polyethers, polyesters, poly(ether-block-amides); or a copolymer, blend, or derivative of any two or more of the foregoing. Exemplary polymers according to the present disclosure include, but are not limited to, a poly(ether-block-amide) (PEBA) (e.g., a block copolymer comprised of a polyamide segment (e.g., polyamide 6 (PA6), or polyamide 6,6 (PA6,6), or polyamide 6,9 (PA6,9), or polyamide 6,10 (PA6,10), or polyamide 6,12 (PA6,12) or polyamide 11 (PA11), or polyamide 12 (PA12)), a polyether segment (e.g., polyoxymethylene (POM), or polyethylene glycol (PEG), or polypropylene glycol (PPG), or polytetramethylene glycol (PTMG)), and may include a polyester chain extender in some grades (e.g., poly(ethylene adipate) (PEA)). Various other polymers may be implemented.

Any non-fluorinated polymeric resin can be used to prepare the non-fluorinated polymeric resin blend according to the present disclosure. Of particular relevance to the present disclosure are blends of non-crosslinked PEBA resins, and thus in various embodiments, can consist of PEBA, can consist essentially of PEBA, or can comprise PEBA. Typically, PEBA resins can be provided in a variety of different forms, for example, in the forms of solid pellets, powders, granules, dispersions, solutions, gels, and the like. In certain embodiments, the tearable tubings are prepared using a blend of medical extrusion grade PEBA resin pellets. The type of PEBA resin that is utilized in certain embodiments can vary and may include PEBA medical extrusion grade pellets of different compositions (e.g., different durometer hardness), as a blend of two, three, or more PEBA copolymer resin grades, or as a blend that includes a PEBA copolymer resin grade. The PEBA resin blends utilized in certain embodiments may also be blended or compounded with other polymeric components to tailor the final properties of the resulting non-crosslinked tearable tube for certain applications. Exemplary medical extrusion grade PEBA resins suitable for use according to the present disclosure are commercially available as PEBAX® 7433 SA 01 MED, PEBAX® 7233 SA 01 MED, PEBAX® 7033 SA 01 MED, PEBAX® 6333 SA 01 MED, PEBAX® 5533 SA 01 MED, PEBAX® 4533 SA 01 MED, PEBAX® 4033 SA 01 MED, PEBAX® 3533 SA 01 MED, PEBAX® 2533 SA 01 MED, and PEBAX® MV 1074 SA 01 MED manufactured by Arkema, Inc, or VESTAMID® Care ME71, VESTAMID® Care ME62, VESTAMID® Care ME55, VESTAMID® Care ME47, VESTAMID® Care ME40, and VESTAMID® Care ME26 manufactured by Evonik Corporation. However, it is to be understood that the composition of the tearable tubings provided herein are not limited to PEBA resins and may be prepared using one or more of the polymeric resins described herein in addition to PEBA, or instead of PEBA.

According to various embodiments, the method employed to blend the non-fluorinated polymeric resins utilized to prepare the tearable heat shrink tubes can vary to influence the characteristics of the final tearable heat shrink tube. For example, two, three, or more resins can be combined in a container and thoroughly mixed by hand or tumbled at room temperature until a homogeneous resin blend is obtained. This is referred to as a ‘dry blending process’ that is relatively simple and allows for advantageous properties of the two, three, or more resins to be realized once extruded into a tubular form (e.g., an input tube formed in a single screw extrusion process). Dry blending provides the lowest amount of mixing possible when preparing a polymeric resin blend and can have certain advantages and/or disadvantages. Depending on the target particular polymeric resins being blended, lower degrees of mixing can produce a final phase separated blend with large phase domains. In some cases, as disclosed herein, this leads to an increase in the tearability and attainable recovery ratio of the final tearable heat shrink tube. In other cases, more thorough mixing is required to obtain a desirable degree of tearability and attainable recovery ratio in the final tearable heat shrink tube. To accomplish this, a twin-screw extrusion process can be employed to vigorously mix the two, three, or more resins utilized in the non-fluorinated polymeric resin blend. Using a twin-screw extrusion process results in a blend “premix” or “compound” that can be subsequently utilized in a single-screw extrusion process to prepare an input tube. The input tube prepared using the compounded resin blend can then be put through a secondary expansion process to fabricate a tearable heat shrink tube according to the present disclosure. This higher degree of mixing tends to result in smaller phase domains and tends to reduce the tearability and attainable recovery ratio of the final tearable heat shrink tube for certain resin blends, as disclosed herein.

In some embodiments, one or more additives can be incorporated within the bulk of the tubing walls, and/or applied upon the inner diameter and/or outer diameter surface. In some such embodiments, the one or more additives can be distributed (e.g., substantially uniformly) throughout the wall thickness and length of the tubing. In some embodiments, the one or more additives may include a lubricant, e.g., such as a thermally stable extrusion process lubricant. In certain embodiments, the lubricant may be a pentaerythritol ester, such as GLYCOLUBE® from Azelis Americas, LLC, for example. In some embodiments, the one or more additives may include a radiopaque filler (e.g., an inorganic radiocontrast agent) to assist in medical procedures that utilize fluoroscopy for navigation of a medical device within the body. In certain embodiments, the radiopaque filler may be barium sulfate (BaSO4), bismuth subcarbonate (Bi₂O₂CO₃), bismuth oxychloride (BiOCl), bismuth trioxide (Bi₂O₃), or tungsten (W), for example. In some embodiments, the one or more additives may include a pigment to provide a desired color of the final PEBA heat shrink tube. In some embodiments, other additives such as inert fillers, stabilizers (e.g., radiation stabilizers, antioxidants, etc.), conductive fillers, anti-tack agents and antimicrobials may be included to produce desired functionality of the final tearable PEBA tube for specific applications. The amount of additive that can be contained in the final tearable PEBA tube is not particularly limited. In various embodiments, for example, the one or more additives (e.g., lubricant, pigment, filler, etc.) may be included in an amount in the range of about 0.1% to about 80%, or about 1% to about 30%, or about 5% to about 20% by weight based on the total weight of the tearable PEBA tube. In other embodiments, the tearable PEBA tube may not include any additives therein.

According to various embodiments, tearable heat shrink tubes exhibit unique properties and unique combinations of properties, as will be outlined further herein. Generally, a heat shrink tubing is a shrinkable tubing prepared via expansion of a polymeric (“input”) tubing (e.g., an extruded tubing) to give the heat shrink tubing (also referred to herein as an “expanded” form). In response to heating the heat shrink tubing in expanded form, the heat shrink tubing “shrinks” to a size that is equivalent to (or close to) its original/input size, commonly referred to as its “recovered” size. The composition and overall size of a tearable heat shrink tubing according to the present disclosure can vary widely and is not particularly limited. A heat shrink tubing can be defined, e.g., by measurable properties such as its inner diameter (“ID”) either after expansion (also referred to herein as “expanded inner diameter” (IDₑ)) or after recovery (also referred to herein as “recovered inner diameter” (IDᵣ)), its length (L), its change in length upon recovery (e.g., its percent change in length upon recovery, ΔL), its average wall thickness, its wall thickness concentricity (also referred to herein as percent concentricity or simply as concentricity), its expansion ratio (ER), its recovery ratio (RR), and its percent change in inner diameter upon recovery (ΔID). Such properties can be defined using the following equations:

$$Expansion ratio = ER = \frac{{ID}_{e}}{{ID}_{o}} \qquad ( 1 )$$

$$Recovery ratio = RR = \frac{{ID}_{e}}{{ID}_{r}} \qquad ( 2 )$$

$$\% Change in Length = Δ L = \frac{{L}_{r} - {L}_{e}}{{L}_{e}} ( 100 ) \qquad ( 3 )$$

$$\% Change of Inner Diameter = Δ ID = \frac{{ID}_{e} - {ID}_{r}}{{ID}_{e}} ( 100 ) \qquad ( 4 )$$

$$\% Concentricity = \frac{{wt}_{min}}{w {t}_{max}} ( 100 ) \qquad ( 5 )$$

In these equations, Lₑ and Lᵣ are the length of the tearable heat shrink tubing (in expanded form) and the length of the “recovered” (e.g., heat-shrunk) tubing, respectively. IDₒ refers to the original internal diameter (ID) of the input tube (e.g., the tube before it is expanded and then subsequently “shrunk”); IDₑ refers to the internal diameter (ID) of the expanded heat shrink tubing; and IDᵣ refers to the internal diameter (ID) of the recovered (e.g., heat shrunk) tube. Values needed for determination of percent concentricity are the minimum wall thickness and the maximum wall thickness of the tubular walls, defined as wtₘᵢₙ and wtₘₐₓ, respectively. RR, ΔL, and ΔID can be evaluated under any recovery conditions (e.g., time, temperature, and method of heat application), though the time and temperature at which an expanded tube is recovered must be specified as this can influence the observed extent of recovery (e.g., an expanded tube that is exposed to a lower temperature and/or for a shorter time may not recover to its full capability). Percent concentricity can be evaluated in the expanded or recovered state. Concentricity is a measure of wall thickness uniformity, and the concentricity value can influence performance in certain applications in both states. As used herein, the above parameters were calculated as follows.

The percent change in length (ΔL), also referred to herein as longitudinal change, is determined in the following manner. Prior to placing the tearable heat shrink tubing into the oven for unrestricted recovery, the expanded tubing is cut to a length of 3.0 inches using a verified ruler. The 3.0-inch specimen length is carefully cut from the tearable heat shrink tubing so as to ensure there are no burs or other deformities present, and that they are perpendicular to the longitudinal axis of the tubing. After the unrestricted recovery process at a specified temperature, the tubing length is re-measured using a verified ruler to the nearest 1/32nd of an inch to determine the amount of shrinkage or growth that has occurred during the process. For example, the expanded length is subtracted from the recovered length and divided by the expanded length, then this quantity is multiplied by 100 to give the overall percent change in length (ΔL) resulting from recovery. Typically, ΔL is measured to be in the range of about +/−10% (e.g., the length changes by less than about 10% upon recovery). In various embodiments, the longitudinal change is measured to be in the range of +/−9%, or +/−5%, or +/−2%. In certain embodiments, longitudinal change has been averaged at 2% or less.

The recovery ratio (RR), percent change in inner diameter (ΔID), and percent concentricity is determined in the following manner. Three 3.0-inch-long specimens are cut from the expanded tubing and their expanded ID and wall thickness is measured using verified measurement tools. Multiple wall thickness measurements must be taken to accurately determine the percent concentricity (e.g., the wall thickness uniformity) of the tubular walls. The minimum wall thickness measurement taken on the expanded tube is divided by the maximum wall thickness measurement taken on the expanded tube, and then multiplied by 100 to give the percent concentricity of the expanded tube. The specimens are then placed into an oven set at a specified temperature for 5 minutes. After exposing each tearable heat shrink tubing specimen to a specified recovery temperature for 5 minutes, the tubing is removed from the oven and allowed to cool to ambient temperature. This subjects the expanded heat shrink tubing to an unrestricted recovery process. After cooling to ambient temperature, the recovered ID and wall thickness is measured using verified measurement tools. The expanded tubing ID is divided by the recovered tubing ID to calculate the recovery ratio (RR) of the heat shrink tube under the specified recovery conditions (e.g., recovery temperature and time). Subsequently, the percent inner diameter change of the heat shrink tubing is calculated by subtracting the expanded tubing ID from the recovered tubing ID and dividing by the expanded tubing ID, then multiplying this quantity by 100 to give the overall percent change in inner diameter (ΔID). The minimum wall thickness measurement taken on the recovered tube is divided by the maximum wall thickness measurement taken on the recovered tube, and then multiplied by 100 to give the percent concentricity of the recovered tube.

The sizes of tearable heat shrink tubes within the scope of this disclosure (e.g., length, diameter (e.g., expanded inner diameter, ID), and average wall thickness) are not particularly limited. For example, the length of tubes described herein can vary from individually sized units (e.g., in some embodiments, on the order of 0.1 inches to 120 inches for catheter or medical device component manufacturing) to lengths that can readily be transported and further cut into individually-sized units to large-scale production lengths (e.g., on the order of hundreds of feet and the like). The diameters of tubes described herein can vary, in particular, depending upon the application for which the tubing is intended. Certain expanded IDs of the tubes described herein, particularly for catheter and medical device uses, can range from about 0.005 inches to about 1.5 inches (e.g., about 0.01 inches to about 0.7 inches or about 0.015 inches to about 0.5 inches), although tubes having expanded IDs outside this range are also encompassed by the present disclosure, particularly in the context of applications in different fields.

By “tearable” as used herein is meant that the tube can be readily torn/peeled apart in the longitudinal direction (e.g., so as to be removed from an underlying material after use in certain embodiments). This tearability/peelability can advantageously allow for the tubing to be provided, used, and removed, in some embodiments, in the absence of any scoring, break lines, indentations, embedded objects, or perforations along the length of the tubing. In certain embodiments, a small score/nick at the end of a length of tubing can allow one to peel the tubing for a significant length, including the full length of the tubing, providing two substantially equal halves of tubing following complete peeling of the length of tubing. The disclosed tubing can, in some embodiments, exhibit one or more of complete, straight, and even peeling along a given length of the tubing.

In general, the methods by which heat shrink tubes are prepared can vary. Generally, the desired resin or resins, such as the non-fluorinated polymeric resin blends described herein, are converted into a tubular form via extrusion and then mechanically expanded. The means by which these steps are conducted can vary, as will be described herein.

A resin (e.g., such as a PEBA resin) may be formed into a tube by subjecting the resin to extrusion. Extrusion generally comprises placing the desired resin or resins into an extruder (e.g., a single screw, twin screw, or quad screw melt extruder). Within the extruder, the resin or resins are heated, compressed, and forced through an annular die set, creating a tube. The annular die set (also referred to herein as “tooling”) consists of a circular extrusion die and a mandrel which forms the polymer melt into a tubular form as it exits the extruder. Tubes of various diameters, wall thicknesses, and lengths can be produced using the forming methods described herein. The final dimensions of the extruded tubular form can be adjusted and optimized through proper tooling selection along with other parameters in the extrusion step such as temperature, extruder pressure, screw rotation speed, and line speed. These parameters can also be adjusted to achieve an optimized level of mixing when preparing a tubular form comprising a blend of more than one polymeric resin. The tube-forming tooling is fitted to the extrusion head (e.g., the end) of the extruder, which is generally comprised of a hopper, barrel, screw(s), breaker plate, and extrusion head. The screw(s) of the extruder is generally comprised of several sections (e.g., the feed, compression, and metering zones) that can be optimized to provide an effective and consistent extrusion process. The extruder screw(s) can also be geometrically optimized with more or less mixing elements to provide a desired level of mixing when preparing a tubular form comprising a blend of more than one polymeric resin. Generally, there are multiple temperature-controlled zones throughout the extruder, each of which can be adjusted and optimized to produce tubular forms of desired dimension and quality. In some embodiments, tubing having a relatively uniform wall thickness (e.g., high percent concentricity) is provided. In some embodiments, the extruded input tubing can be subjected to low doses of radiation such as e-beam or gamma to induce branching and in some cases a low degree of crosslinking.

The extruded tubular form can then be radially expanded (e.g., by mechanical means) to provide an expanded tube, e.g., a heat shrink tube (e.g., a tubing which decreases in diameter when heated). The expansion of the input tubing (e.g., the initial extruded tubular form) can be conducted in-line with extrusion or off-line (e.g., conducted independently of and/or secondary to the extrusion process). All means for radial expansion of tubing are intended to be encompassed by the present invention. Generally, during the expansion process, the tubing is expanded radially by pressurizing the inside of the tubing, introducing stress into the tube wall. This pressurizing can be conducted by any means capable of providing a differential pressure between the inside and outside of the tubing. Such differential pressure can be created by imposing a pressure above atmospheric pressure on the inside of the tube, imposing a pressure below atmospheric pressure on the outside of the tube, or a combination of the two. The stress induced into the wall of the tube causes it to expand radially, e.g., increase in diameter. The rate of expansion can be controlled so the tube will hold the expanded state and does not recover until subjected to a further heat cycle. The extent to which a tube is expanded depends on the application for which the final heat shrink tubing is intended. The rate and extent to which a tube is expanded depends on the temperature at which the expansion process is conducted. It has been found that the expansion chamber temperature must be carefully controlled to optimize the rate and extent of expansion of the tube. In some embodiments, the tubing is expanded to an inner diameter from about 1.05 times its original (unexpanded) inner diameter to about 10 times its original (unexpanded) inner diameter.

In certain embodiments, tearable PEBA heat shrink tubes prepared according to the present disclosure may be radially expanded using the processes described, for example, in U.S. Pat. No. 9,296,165 to Henson, which is incorporated by reference herein in its entirety. For example, the Henson patent describes a process for the production of thermoplastic polymeric heat shrink tubing using a first fluid in the interior of a tube to expand it and a second fluid exterior to the tube to constrain the expansion within an expansion chamber. In other embodiments, for example, the tubing may be expanded by adjusting the flow rate of the air external to the tube, the chamber temperature, the air pressure within the tube, and the rate at which the tube moves through the expansion chamber. In certain embodiments, the tearable heat shrink tubes of the present disclosure are expanded at elevated temperature through a die using any number of methods known to the art, and subsequently cooled at the die exit. Cooling can be accomplished using fluids such as water, oil, or air. The processing parameters that can be adjusted include, but are not limited to: die type, die diameter and length, die temperature, fluid pressure inside the tube, fluid pressure outside the tube, cooling method, cooling medium type and temperature, expansion rate, tube material, tube ID, tube OD, and tube wall thickness.

In some embodiments, the PEBA tubes are tearable or peelable without having been radially expanded. The PEBA tubes are not heat shrinkable until they have been radially expanded.

In some embodiments, the heat shrink tubes comprise noncross-linked PEBA blends. All PEBA in the heat shrink tubes is noncross-linked.

In some embodiments, the PEBA tubes comprise some cross-linked polymer. For example, the tubes may comprise less than about 2% by weight of a crosslinked polymer. As another example, the tubes may comprise less than about 1% by weight of a cross-linked polymer. As another example, the tubes may comprise less than about 0.5% by weight of a cross-linked polymer.

The disclosed tearable heat shrink tubes can exhibit high recovery ratios; in some embodiments, the disclosed tearable heat shrink tubes can have recovery ratios (RRs) of greater than 1.05:1, or greater than 1.10:1, or greater than 1.2:1, or greater than 1.3:1, or greater than 1.4:1, or greater than 1.5:1, or greater than 1.6:1. As an example, in some embodiments, the tearable heat shrink tubes have a RR between 1.05:1 to 2:1. In some embodiments, the tearable heat shrink tubes can be described based on the reducibility in ID (upon recovery). In some embodiments, the tearable heat shrink tubes are reducible in the ID by between 4% to 37.5%. In some embodiments, the tearable heat shrink tubes are reducible in the ID by between 5% to 40%. Examples of such values include, but are not limited to, tubes reducible in ID by at least 4%, or at least 5%, or at least 6%, or at least 7%, or at least 8%, or at least 9%, or at least 10%, or at least 11%, or at least 12%, or at least 13%, or at least 14%, or at least about 15%, or at least 20%, or at least 25%, or at least 30%; or at least 37.5%, or at least 40%. s

The tearable heat shrink tubes provided herein can be used in various ways. In some embodiments, they are advantageously used in catheter assembly processes, e.g., as a replacement for conventional (e.g., FEP) heat shrink tubings used as manufacturing aids to compress an underlying catheter pre-assembly during heating/reflow. According to the present disclosure, the provided tearable heat shrink tubes (e.g., tearable PEBA heat shrink tubes) can function both as a catheter jacket and as a processing aid/heat shrink to provide sufficient compression during heating/reflow.

As such, the disclosed tearable heat shrink tubes enable a catheter shaft to be manufactured by heating the heat shrink tube as an outer layer (“jacket”) of a catheter shaft pre-assembly, inducing dimensional recovery of the heat shrink tube and allowing for reflow through a reinforcing component (where present) and bonding to the underlying liner without the use of a fusing sleeve (e.g., FEP). Because the polymeric material of the disclosed heat shrink tube is not crosslinked, its viscosity is such that the polymer can easily flow through and encapsulate a reinforcing component (where present) and adhere to the outer surface of the inner liner when heated under an appropriate recovery profile. The selection of an appropriate heating temperature and time (also referred to herein as “heating profile” or “recovery profile”) for a particular non-crosslinked heat shrink outer jacket tube can vary substantially depending on the underlying catheter assembly components and the composition of the non-crosslinked heat shrink tubing. In particular applications, the recovery ratio can also be tailored along with the recovery profile to provide a catheter shaft where the outer jacket only bonds to the outer surface of a reinforcing component, leaving the underlying interstices open to allow for a catheter shaft with increased flexibility. In this way, expansion conditions, recovery ratio, and the recovery profile can be tailored to provide a non-crosslinked PEBA heat shrink tube capable of forming an outer sheath for various different types of catheter structures.

The tearable heat shrink tubes provided herein can be used for a range of applications. In particular applications, tearable heat shrink tubes as provided herein can be applied to an underlying material (e.g., devices, device components, joints, fittings, wires, etc.), and heated (e.g., recovered) to form a covering thereon. Optionally, the recovered heat shrink tube forming a covering over and underlying material or construct can be torn away after initiating a cut on one end of the tearable heat shrink tube. This can be advantageous in various applications where intermittent maintenance to an underlying component is necessary (e.g., an underlying electronic or mechanical component). Accordingly, the present disclosure encompasses materials or objects to which a tube as disclosed herein has been applied. For example, in some embodiments, a covered device (e.g., medical device) comprising a tearable heat shrink tube (e.g., in recovered form) as disclosed herein is provided. Exemplary covered devices include, but are not limited to, medical devices (e.g., catheters, catheter shafts, and catheter shaft components) comprising any of the tubes disclosed herein applied thereto (in expanded/non-recovered and recovered forms). In some embodiments, a covered hypotube (e.g., a laser-cut hypotube) is provided.

According to various embodiments, another application is the use of the tube as a switch (e.g., a one-way switch) that is set based on temperature. The catheter (or other medical device) may comprise a tube at the distal end. The tube may be in an expanded form. The induction of heat to the tube can be used to switch a configuration of the catheter. As an illustrative example, the tube can be disposed at the distal end of the catheter. During use of the catheter (e.g., during a procedure), the tube in its expanded form can serve as a lumen that remains open for aspiration (or other purposes). Heat may be induced to the tube to cause the tube to shrink to reduce the size of the lumen or to close the lumen completely. As an example, the heat may be induced via a remote heat source such as through a resistive heating element. As another illustrative example, the expanded tube disposed on the catheter (e.g., the distal end of the catheter) can be used as a heat/temperature monitor whereby the tube shrinks upon the introduction of heat above a predefined temperature.

In some embodiments, a plurality of tearable heat shrink tubes as provided herein having different durometer hardness (e.g., flexibility) can be joined together (e.g., using tape or adhesive, heat or solvent welding, interference fit, etc.) and placed over a catheter assembly prior to recovery to form a catheter shaft with varying degrees of flexibility and smooth transitions. For example, the plurality of heat shrink tubes can comprise, e.g., two or more, three or more, four or more, five or more, six or more, seven or more, eight or more, nine or more, or ten or more tearable heat shrink tubes with different durometer hardness values. In some embodiments, heat shrink tubes as provided herein having different durometer hardness (e.g., flexibility) can be sequentially placed and recovered over a catheter assembly. The transitions between adjacent heat shrink tubes can be, in some embodiments, smoothed by passing the construct through a heated metallic or polymer-coated die either during or after the forming (e.g., recovery) process is completed to form a catheter shaft with varying degrees of flexibility and smooth transitions. In certain embodiments, tearable heat shrink tubes as provided herein having different durometer hardness (e.g., flexibility) can be sequentially placed and recovered over a catheter assembly; the tubes can optionally be further treated to smooth the transitions between outer jacket sections using a heat shrinkable fusing sleeve to form a catheter shaft with varying degrees of flexibility and smooth transitions.

It is noted that, although certain heat shrink PEBA tubes are known, these tubes comprise predominantly crosslinked PEBA and do not exhibit tearability. Generally, the tearable PEBA heat shrink tubes provided herein are fabricated under carefully controlled conditions during extrusion and expansion to promote tearability and to lock in the entropically unfavorable expanded state. The PEBA tubes of the present disclosure exhibit tearability as extruded (e.g., as the input tube), after expansion (e.g., as the expanded heat shrink tubing before recovery), and in the recovered state (e.g., after unrestricted recovery or after recovering the expanded tube over a substrate). Examples of heat shrinkable crosslinked PEBA tubing are provided, for example, by Cobalt Polymers, TE Connectivity, and in the disclosures of U.S. Pat. No. 7,306,585 to Ross and U.S. Pat. App. Pub. No. 2008/0317991 to Pieslak et al; none of these examples exhibit tearability. Further, none of these examples disclose a PEBA resin blend (e.g., a single wall heat shrinkable tubing comprising a formulation of more than one PEBA resin), and therefore would not exhibit tearability. In particular, the disclosure of Pieslak et al describes a dual wall heat shrinkable construct where one of the layers is substantially crosslinked to “render the assembly dimensionally recoverable” and a second layer that is preferably non-crosslinked to allow for the incorporation of an underlying reinforcing structure upon recovery. It is further noted that the composition of the tearable heat shrink tubes of the present disclosure do not include crosslink-promoting additives as disclosed in Pieslak et al. Crosslinking through chemical means or by irradiation has long been used in the production of heat shrink tubes and films in order to obtain a greater degree of elastic recovery of the expanded part upon heating (e.g., increase attainable recovery ratio and/or recovery force upon heating). Crosslinking a polymer article such as a tube or film effectively increases the molecular weight of the polymer in addition to improving its elastic response to an imposed deformation. This effective increase in molecular weight also results in a marked increase in the viscosity of the polymer (due to the increased probability of interchain entanglements), which also increases the stiffness (e.g., reduces the flexibility) of the material. The increased viscosity due to crosslinking also reduces the ability of the polymer to conform tightly to and bond with an underlying substrate during recovery. Substantially crosslinking a polymer article also renders the tubing unable to flow above the melt temperature and does not allow the material to be reshaped using a fusing sleeve that has a recovery temperature greater than the melting temperature of the crosslinked polymer article (e.g., FEP heat shrink).

It has been documented in the referenced publication of Murray et al. that a low dose of e-beam radiation (e.g., 0 to 10 kGy) predominantly induces a reduction in molecular weight due to irradiation degradation such as chain-scission. Murray et al note that as the delivered e-beam dosage is increased from 10 kGy, Melt Flow Index (MFI) data suggests that changes occur to the material that results in the restriction of flow properties. Their findings show that both branching and crosslinking is induced at e-beam irradiation doses higher than 10 kGy, but e-beam irradiation preferentially induces branching to occur in the soft segment (e.g., the polyether segment) of PEBA. At 200 kGy of e-beam irradiation, an MFI value of 0 g/10 min (e.g., no flow) was reached which Murray et al associates with “the existence of a high concentration of molecular weight in the material from crosslinking”. However, their results show that at 100 kGy the MFI value was around 2.5 g/10 min which suggests that the material has undergone changes that restrict flow (e.g., branching) but is not substantially crosslinked. The formulations utilized in the irradiated tearable heat shrink tubes of the present disclosure do not include crosslink promoters, such as triallyl isocyanurate (TAIC), as utilized in the formulations of the “substantially crosslinked dimensionally recoverable layer” of Pieslak et al. Further, the average surface dose of e-beam radiation utilized in the fabrication of the irradiated tearable heat shrink tubes of the present disclosure was 100 kGy or less, further supporting that these formulations are not substantially crosslinked.

Various non-fluorinated polymeric resin blends can be used to form the tearable tubes. In some embodiments, the non-fluorinated resin blends utilized comprise more than one PEBA resin, however, it is to be understood that the composition of the tearable tubings provided herein are not limited to PEBA resins and may be prepared using one or more of the polymeric resins described herein in addition to PEBA, or instead of PEBA. In certain embodiments, tearable tubes comprising a PEBA resin blend are provided. In other specific embodiments, tearable heat shrink tubes comprising a PEBA resin blend are provided. When referring to the composition of a particular PEBA resin grade, the polyamide or nylon 12 segment (PA12) can be referred to as the “hard segment” or “hard phase”, the polyether or polytetramethylene oxide segment (PTMO) can be referred to as the “soft segment” or “soft phase”, and the polyester or adipic acid segment (present in very low concentrations in particular grades of PEBA resin) acts as a chain extender. The ratio of the polyamide, polyether, and polyester segments of the PEBA resin blend used to prepare the tearable tube can vary greatly without departing from the present disclosure. Different composition ratios (e.g., mole percent (mol %)) of the polyamide hard segment to polyether soft segment influence the physical properties of the supplied resin. Varying the mass ratio (e.g., weight percent (weight %)) of PEBA grades utilized in the blend influences the physical properties of the extruded input tube; and ultimately the final physical properties of the tearable heat shrink tubes of the present invention. This allows for tearable heat shrink tubes of varying tear strength, flexibility, recovery ratio, and other physical properties to be produced.

### Experimental

Aspects of the present invention are more fully illustrated by the following examples, which are set forth to illustrate certain aspects of the present invention and are not to be construed as limiting thereof. Although the examples shown pertain to tearable heat shrink tubing comprising blends of PEBA resins specifically, it is understood that tearable heat shrink tubing comprised of other non-fluorinated polymeric materials would benefit according to the present invention.

### Evaluation of Tearability (Tear Index)

Example tubes prepared as described herein were subjected to a tear test to evaluate tearability. A razor blade was used to initiate a cut between 0.25 to 0.50 inches in length along the central line of the longitudinal axis on one end of a 10-inch length of sample. Both ‘tear tabs’ were grasped by hand and the tube was torn apart at a moderate rate such that the tear was complete within 5 seconds. The two resulting halves of the tube were weighed to the nearest 0.01 mg and recorded. This provides two mass values that allow for calculation of the Tear Index (TI), determined by the quotient of the smaller mass to larger mass. The Tear Index is a measurable value that quantifies how evenly the initial cut propagates along the length of the tube during the tear test. Tearability was evaluated in triplicate, the average of which is shown in Table 3.

### Evaluation of Gel Content & Swelling Index

The degree of crosslinking of a polymeric material can be determined by measuring the Gel Content. The gel content of a polymeric material is a measure of the amount of insoluble crosslinked material present. The gel content can be evaluated, for example, by placing approximately 15 mg of sample material into a vial along with 20 mL of hexafluoroisopropanol (HFIP) and allowing the material to shake on a standard laboratory shaker for 72 hours. Then, the prepared samples can be filtered through filter paper of known dry mass. After filtering the samples, fresh solvent can be used to rinse the samples to ensure no soluble portion of the original sample is left behind. The combined sample and filter paper can be thoroughly dried in a vacuum oven so that the final dry mass of sample (i.e., dry gel portion) and filter paper can be measured. The mass of the original sample placed into the vial and the mass of the dry gel portion can be used to determine the gel content of the original sample, e.g., the percent gel content of the original sample.

$$\% Gel Content = ( \frac{Dried Gel Mass}{Original Sample Mass} ) \times 100 \qquad ( 6 )$$

### Melt Flow Index (MFI)

Melt Flow Index, also commonly referred to as Melt Flow Rate (MFR), can be used to measure the ability of a polymeric material to flow above the melt temperature. It can also be used as a tool to infer macromolecular changes in a polymeric material resulting from ionizing radiation, as shown in the referenced publication from Murray et al. Test methods useful for the determination of MFI or MFR are ASTM D1238 Standard Test Method for Melt Flow Rates of Thermoplastics by Extrusion Plastometer. It is important to note that if a material is substantially crosslinked the MFI of that material will be zero, given that substantially crosslinked materials do not flow above the melt temperature. Generally, a higher MFI represents a material with a lower melt viscosity and a greater ability to flow above the melt temperature. Conversely, a lower MFI represents a material with a higher melt viscosity and a lesser ability to flow above the melt temperature. For a given neat (e.g., unfilled) polymeric system the MFI can be influenced by several factors including molecular weight and macromolecular architecture.

### Preparation of Examples

The following Examples and Comparative Examples, tearable and non-tearable heat shrink tubes of varying composition were prepared using various methods according to the present disclosure.

### Example 1

A non-fluorinated polymeric resin blend was prepared by hand mixing and tumbling (e.g., dry blending) 80% by weight of commercially available PEBAX® 7233 SA 01 MED (Arkema, Inc) resin pellets and 20% by weight of PEBAX® 5533 SA 01 MED (Arkema, Inc) resin pellets. The blended pellets were extruded into a tubular form by utilizing a single screw extruder having a barrel diameter of 18 mm, a screw rotation of about 3 rpm, a die temperature of approximately 360° F., and an annular die set that provided a draw down ratio (DDR) around 10. After exiting the annular die set, the tube was passed through a chilled water bath to sufficiently quench the tubing and set the final tubular dimensions. An input tube having an inner diameter of about 0.044″ and an average wall thickness of about 0.0100″ was obtained.

The prepared input tube was then expanded by pressurizing the inner diameter of the tube with compressed air as it is passed through a heated expansion die. The heated expansion die has openings along its inner surface that allows for pressurized air to circulate between the outer surface of the input tube and the inner surface of the expansion die to maintain a specified expanded diameter. The processing parameters of expansion air pressure applied to the ID of the input tubing, temperature of the pressurized expansion air applied to the ID of the input tubing, expansion die air pressure, expansion die air temperature, expansion die air flowrate, linear tube throughput, cooling air temperature, and cooling air flowrate were all adjusted to give a tearable heat shrink tube according to the present disclosure.

In particular, the tearable heat shrink tube of Example 1 was expanded using an expansion die temperature of about 310° F. to about 330° F., an expansion air pressure of about 81 psi, a die air flowrate of about 2.2 cubic feet per minute (cfm) and a linear tube throughput of around 3 feet per minute (fpm).

The dimensional attributes of inner diameter and wall thickness of the expanded form of Example 1 were measured. Three specimens of the expanded tearable heat shrink tubing of Example 1 were cut and exposed to a recovery temperature for 5 minutes in a standard laboratory gravity convection oven. After allowing the lengths to cool to ambient temperature, the inner diameter, wall thickness, and length after heating were measured. The measured dimensional attributes before heating (e.g., when the tube is in expanded form), after heating (e.g., after being recovered), and the calculated recovery properties of the tearable heat shrink tubes are summarized in Table 2. Three 10-inch-long specimens of the expanded tearable heat shrink tubing of Example 1 were cut and evaluated for tearability. The Tear Index results are shown in Table 3.

### Example 2

A non-fluorinated polymeric resin blend was prepared by dry blending 50% by weight of commercially available PEBAX® 7233 SA 01 MED (Arkema, Inc) resin pellets and 50% by weight of PEBAX® 5533 SA 01 MED (Arkema, Inc) resin pellets. An input tube was prepared from this blend using the same methods and conditions provided in Example 1. An input tube having an inner diameter of about 0.040″ and an average wall thickness of about 0.0106″ was obtained.

The prepared input tube was then expanded using the same methods and conditions as Example 1, except an expansion die temperature of about 300° F. to about 320° F., an expansion air pressure of about 77 psi, and a die air flowrate of about 1.8 cfm was used.

### Example 3

A non-fluorinated polymeric resin blend was prepared by dry blending 20% by weight of commercially available PEBAX® 7233 SA 01 MED (Arkema, Inc) resin pellets and 80% by weight of PEBAX® 5533 SA 01 MED (Arkema, Inc) resin pellets. An input tube was prepared from this blend using the same methods and conditions provided in Example 1. An input tube having an inner diameter of about 0.038″ and an average wall thickness of about 0.0117″ was obtained.

The prepared input tube was then expanded using the same methods and conditions as Example 1, except an expansion die temperature of about 295° F. to about 310° F., an expansion air pressure of about 76 psi, and a die air flowrate of about 2.0 cfm was used.

### Example 4

A non-fluorinated polymeric resin blend was prepared by dry blending 80% by weight of commercially available PEBAX® 5533 SA 01 MED (Arkema, Inc) resin pellets and 20% by weight of PEBAX® 3533 SA 01 MED (Arkema, Inc) resin pellets. An input tube was prepared from this blend using the same methods and conditions provided in Example 1. An input tube having an inner diameter of about 0.040″ and an average wall thickness of about 0.0111″ was obtained.

The prepared input tube was then expanded using the same methods and conditions as Example 1, except an expansion die temperature of about 295° F. to about 310° F., an expansion air pressure of about 41 psi, and a die air flowrate of about 2.1 cfm was used.

### Example 5

A non-fluorinated polymeric resin blend was prepared by dry blending 50% by weight of commercially available PEBAX® 5533 SA 01 MED (Arkema, Inc) resin pellets and 50% by weight of PEBAX® 3533 SA 01 MED (Arkema, Inc) resin pellets. An input tube was prepared from this blend using the same methods and conditions provided in Example 1. An input tube having an inner diameter of about 0.039″ and an average wall thickness of about 0.0106″ was obtained.

The prepared input tube was then expanded using the same methods and conditions as Example 1, except an expansion die temperature of about 280° F. to about 290° F., an expansion air pressure of about 31 psi, and a die air flowrate of about 2.4 cfm was used.

### Example 6

A non-fluorinated polymeric resin blend was prepared by dry blending 20% by weight of commercially available PEBAX® 5533 SA 01 MED (Arkema, Inc) resin pellets and 80% by weight of PEBAX® 3533 SA 01 MED (Arkema, Inc) resin pellets. An input tube was prepared from this blend using the same methods and conditions provided in Example 1. An input tube having an inner diameter of about 0.039″ and an average wall thickness of about 0.0110″ was obtained.

The prepared input tube was then expanded using the same methods and conditions as Example 1, except an expansion die temperature of about 250° F. to about 255° F., an expansion air pressure of about 31 psi, and a die air flowrate of about 2.2 cfm was used.

### Example 7

A non-fluorinated polymeric resin blend was prepared by dry blending 80% by weight of commercially available PEBAX® 7233 SA 01 MED (Arkema, Inc) resin pellets and 20% by weight of PEBAX® 3533 SA 01 MED (Arkema, Inc) resin pellets. An input tube was prepared from this blend using the same methods and conditions provided in Example 1. An input tube having an inner diameter of about 0.041″ and an average wall thickness of about 0.0112″ was obtained.

The prepared input tube was then expanded using the same methods and conditions as Example 1, except an expansion die temperature of about 300° F. to about 310° F., an expansion air pressure of about 92 psi, and a die air flowrate of about 2.4 cfm was used.

### Example 8

A non-fluorinated polymeric resin blend was prepared by dry blending 50% by weight of commercially available PEBAX® 7233 SA 01 MED (Arkema, Inc) resin pellets and 50% by weight of PEBAX® 3533 SA 01 MED (Arkema, Inc) resin pellets. An input tube was prepared from this blend using the same methods and conditions provided in Example 1. An input tube having an inner diameter of about 0.038″ and an average wall thickness of about 0.0106″ was obtained.

The prepared input tube was then expanded using the same methods and conditions as Example 1, except an expansion die temperature of about 250° F. to about 265° F., an expansion air pressure of about 48 psi, and a die air flowrate of about 2.5 cfm was used.

### Example 9

A non-fluorinated polymeric resin blend was prepared by dry blending 20% by weight of commercially available PEBAX® 7233 SA 01 MED (Arkema, Inc) resin pellets and 80% by weight of PEBAX® 3533 SA 01 MED (Arkema, Inc) resin pellets. An input tube was prepared from this blend using the same methods and conditions provided in Example 1. An input tube having an inner diameter of about 0.040″ and an average wall thickness of about 0.0103″ was obtained.

The prepared input tube was then expanded using the same methods and conditions as Example 1, except an expansion die temperature of about 250° F. to about 255° F., an expansion air pressure of about 25 psi, and a die air flowrate of about 2.1 cfm was used.

### Example 10

A non-fluorinated polymeric resin blend was prepared by melt compounding 20% by weight of commercially available PEBAX® 7233 SA 01 MED (Arkema, Inc) resin pellets and 80% by weight of PEBAX® 3533 SA 01 MED (Arkema, Inc) resin pellets in a twin-screw extruder having an 18 mm barrel, a die temperature of 360° F., and a screw rotation speed of 200 rpm. An input tube was prepared from this compounded blend using the same methods and conditions provided in Example 1. An input tube having an inner diameter of about 0.040″ and an average wall thickness of about 0.0100″ was obtained. The prepared input tube was then expanded using the same methods and conditions as Example 9.

### Example 11

A non-fluorinated polymeric resin blend was prepared by melt compounding 40% by weight of commercially available PEBAX® 7233 SA 01 MED (Arkema, Inc) resin pellets and 60% by weight of PEBAX® 3533 SA 01 MED (Arkema, Inc) resin pellets in a twin-screw extruder using the same conditions as the compounded blend of Example 10. An input tube was prepared from this compounded blend using the same methods and conditions provided in Example 1. An input tube having an inner diameter of about 0.040″ and an average wall thickness of about 0.0100″ was obtained. The prepared input tube was then expanded using the same methods and conditions as Example 9, except an expansion air pressure of about 33 psi was used.

### Example 12

Example 12 was prepared by utilizing the input tube of Example 10, except before expansion the input tubing was dosed with 100 kGy of electron-beam irradiation in a double pass process. The irradiated input tube was then expanded using the same methods and conditions as Example 9.

### Example 13

Example 13 was prepared by utilizing the input tube of Example 5, except before expansion the input tubing was dosed with 100 kGy of electron-beam irradiation in a double pass process. The irradiated input tube was then expanded using the same methods and conditions as Example 5, except an expansion pressure of 54 psi was used.

### Example 14

Example 14 was prepared by utilizing the input tube of Example 4, except before expansion the input tubing was dosed with 100 kGy of electron-beam irradiation in a double pass process. The irradiated input tube was then expanded using the same methods and conditions as Example 4.

### Example 15

A non-fluorinated polymeric resin blend was prepared by dry blending 40% by weight of commercially available PEBAX® 7233 SA 01 MED (Arkema, Inc) resin pellets, 40% by weight of PEBAX® 3533 SA 01 MED (Arkema, Inc) resin pellets and 20% by weight of PEBAX® 5533 SA 01 MED (Arkema, Inc) resin pellets. An input tube was prepared from this blend using the same methods and conditions provided in Example 1. An input tube having an inner diameter of about 0.052″ and an average wall thickness of about 0.0057″ was obtained. The prepared input tube was then expanded using the same methods and conditions as Example 9, except an expansion air pressure of about 38 psi was used.

### COMPARATIVE EXAMPLES

### Comparative Example 1

An input tube was prepared using commercially available PEBAX® 7233 SA 01 MED (Arkema, Inc) resin pellets using the same methods and conditions provided in Example 1. An input tube having an inner diameter of about 0.048″ and an average wall thickness of about 0.0145″ was obtained.

The prepared input tube was then expanded using the same methods and conditions as Example 1, except an expansion die temperature of about 305° F. to about 310° F., and an expansion air pressure of about 180 psi was used.

### Comparative Example 2

An input tube was prepared using commercially available PEBAX® 5533 SA 01 MED (Arkema, Inc) resin pellets using the same methods and conditions provided in Example 1. An input tube having an inner diameter of about 0.051″ and an average wall thickness of about 0.0160″ was obtained.

The prepared input tube was then expanded using the same methods and conditions as Example 1, except an expansion die temperature of about 265° F. to about 275° F., and an expansion air pressure of about 120 psi was used.

### Comparative Example 3

An input tube was prepared using commercially available PEBAX® 3533 SA 01 MED (Arkema, Inc) resin pellets using the same methods and conditions provided in Example 1. An input tube having an inner diameter of about 0.062″ and an average wall thickness of about 0.0150″ was obtained.

The prepared input tube was then expanded using the same methods and conditions as Example 1, except an expansion die temperature of about 180° F. to about 210° F., an expansion air pressure of about 40 psi, and a die air flowrate of about 1.7 cfm was used.

### Comparative Example 4

A non-fluorinated polymeric resin blend was prepared by melt compounding 90% by weight of commercially available PEBAX® 2533 SA 01 MED (Arkema, Inc) resin pellets and 10% by weight of PEBAX® 7233 SA 01 MED (Arkema, Inc) resin pellets in a twin-screw extruder using the same conditions as the compounded blend of Example 10. An input tube was prepared from this compounded blend using the same methods and conditions provided in Example 1. An input tube having an inner diameter of about 0.062″ and an average wall thickness of about 0.0140″ was obtained.

### Comparative Example 5

A commercially available PEBA heat shrink tube manufactured by Cobalt Polymers (Part No. P2-100-0025-CLR) that is marketed as a crosslinked 72 durometer Shore D PEBA heat shrink tube was purchased from Chamfr.

### Comparative Example 6

A commercially available PEBA heat shrink tube manufactured by Cobalt Polymers (Part No. P2-060-003-40-CLR) that is marketed as a crosslinked 40 durometer Shore D PEBA heat shrink tube was purchased from Chamfr.

### Cumulative Results

Table 1 summarizes the composition, blend method, and e-beam irradiation dose utilized to fabricate the tearable heat shrink tubes of Examples 1-15. Table 2 summarizes the composition of the prepared heat shrink tubes of Comparative Examples 1-4.

TABLE 1
Composition of Tearable Heat Shrink Example Tubes

| Resin | Pebax | Pebax | Pebax | Blend | Irradiation | Recovery |
|---|---|---|---|---|---|---|
| (weight %) | 7233 | 5533 | 3533 | Method | Dose (kGy) | Ratio |
| Ex. 1 | 80 | 20 | — | HB | 0 | 1.33 |
| Ex. 2 | 50 | 50 | — | HB | 0 | 1.36 |
| Ex. 3 | 20 | 80 | — | HB | 0 | 1.55 |
| Ex. 4 | — | 80 | 20 | HB | 0 | 1.42 |
| Ex. 5 | — | 50 | 50 | HB | 0 | 1.58 |
| Ex. 6 | — | 20 | 80 | HB | 0 | 1.69 |
| Ex. 7 | 80 | — | 20 | HB | 0 | 1.38 |
| Ex. 8 | 50 | — | 50 | HB | 0 | 1.73 |
| Ex. 9 | 20 | — | 80 | HB | 0 | 2.23 |
| Ex. 10 | 20 | — | 80 | TSB | 0 | 2.01 |
| Ex. 11 | 40 | — | 60 | TSB | 0 | 1.98 |
| Ex. 12 | 20 | — | 80 | TSB | 100 | 2.81 |
| Ex. 13 | — | 50 | 50 | HB | 100 | 2.32 |
| Ex. 14 | — | 80 | 20 | HB | 100 | 1.92 |
| Ex. 15 | 40 | 20 | 40 | HB | 0 | 1.36 |

TABLE 2
Composition of Prepared Heat Shrink Comparative Example Tubes

| Resin | Pebax | Pebax | Pebax | Pebax | Blend | Irradiation | Recovery |
|---|---|---|---|---|---|---|---|
| (weight %) | 7233 | 5533 | 3533 | 2533 | Method | Dose (kGy) | Ratio |
| Comp. 1 | 100 | — | — | — | N/A | 0 | 1.16 |
| Comp. 2 | — | 100 | — | — | N/A | 0 | 1.23 |
| Comp. 3 | — | — | 100 | — | N/A | 0 | 1.27 |
| Comp. 4 | 10 | — | — | 90 | TSB | 0 | **** |

TABLE 3
Dimensional Attributes of the Examples

| | | Expanded | | Recovered | | | |
|---|---|---|---|---|---|---|---|
| | Recovery Temp | ID | Wall | ID | Wall | | LC |
| Sample | [° F.] | [in] | [in] | [in] | [in] | RR | [%] |
| Ex. 1 | 340 | 0.100 | 0.0044 | 0.075 | 0.0062 | 1.33 | −5.2% |
| Ex. 2 | 330 | 0.089 | 0.0059 | 0.065 | 0.0069 | 1.36 | −3.1% |
| Ex. 3 | 330 | 0.094 | 0.0063 | 0.061 | 0.0085 | 1.55 | −3.5% |
| Ex. 4 | 320 | 0.087 | 0.0062 | 0.062 | 0.0082 | 1.42 | −4.9% |
| Ex. 5 | 310 | 0.094 | 0.0053 | 0.060 | 0.0079 | 1.58 | −6.6% |
| Ex. 6 | 300 | 0.094 | 0.0056 | 0.056 | 0.0088 | 1.69 | −3.8% |
| Ex. 7 | 320 | 0.109 | 0.0047 | 0.079 | 0.0064 | 1.38 | −6.3% |
| Ex. 8 | 310 | 0.094 | 0.0055 | 0.055 | 0.0084 | 1.73 | −1.0% |
| Ex. 9 | 300 | 0.095 | 0.0050 | 0.043 | 0.0094 | 2.23 | −2.4% |
| Ex. 10 | 300 | 0.095 | 0.0046 | 0.047 | 0.0082 | 2.01 | −2.4% |
| Ex. 11 | 300 | 0.098 | 0.0044 | 0.049 | 0.0079 | 1.98 | −2.8% |
| Ex. 12 | 300 | 0.109 | 0.0037 | 0.039 | 0.0097 | 2.81 | **** |
| Ex. 13 | 300 | 0.097 | 0.0037 | 0.042 | 0.0095 | 2.32 | **** |
| Ex. 14 | 300 | 0.090 | 0.0055 | 0.047 | 0.0096 | 1.92 | **** |
| Ex. 15 | 300 | 0.092 | 0.0034 | 0.067 | 0.0045 | 1.36 | −1.6% |
| Comp. 1 | 320 | 0.088 | 0.0097 | 0.075 | 0.0111 | 1.16 | 0.0% |
| Comp. 2 | 300 | 0.087 | 0.0101 | 0.071 | 0.0120 | 1.23 | −1.3% |
| Comp. 3 | 260 | 0.089 | 0.0113 | 0.070 | 0.0141 | 1.27 | −5.0% |
| Comp. 5 | 340 | 0.108 | 0.0016 | 0.079 | 0.0022 | 1.37 | −6.1% |
| Comp. 6 | 340 | 0.067 | 0.0014 | 0.033 | 0.0023 | 2.02 | −2.8% |

TABLE 4
Recovery Ratio and Tear Index of the Examples

| Sample | RR | TI |
|---|---|---|
| Ex. 1 | 1.33 | 0.79 |
| Ex. 2 | 1.36 | 0.94 |
| Ex. 3 | 1.55 | 0.88 |
| Ex. 4 | 1.42 | 0.91 |
| Ex. 5 | 1.58 | 0.93 |
| Ex. 6 | 1.69 | 0.86 |
| Ex. 7 | 1.38 | 0.92 |
| Ex. 8 | 1.73 | 0.96 |
| Ex. 9 | 2.23 | 0.85 |
| Ex. 10 | 2.01 | 0.69 |
| Ex. 11 | 1.98 | 0.77 |
| Ex. 12 | 2.81 | 0.68 |
| Ex. 13 | 2.32 | 0.91 |
| Ex. 14 | 1.92 | **** |
| Ex. 15 | 1.36 | **** |
| Comp. 1 | 1.16 | 0.01 |
| Comp. 2 | 1.23 | 0.02 |
| Comp. 3 | 1.27 | 0.03 |
| Comp. 4 | **** | 0.02 |
| Comp. 5 | 1.37 | **** |
| Comp. 6 | 2.02 | **** |

TABLE 5
Gel Content of Examples

| Sample | Gel Content (%) |
|---|---|
| Ex. 4 | 32 |
| Ex. 14 | 20 |
| Ex. 10 | 31 |
| Ex. 12 | 53 |
| Comp. 5 | 100 |
| Comp. 6 | 85 |

Many modifications and other embodiments of the invention will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A heat shrink tubing comprising PEBA, wherein the heat shrink tubing has a recovery ratio (RR) greater than about 1.05:1, and wherein the expanded heat shrink tubing is at least partially tearable.

2. The heat shrink tubing of claim 1, consisting essentially of non-crosslinked PEBA.

3. The heat shrink tubing of claim 1, comprising less than 60% by weight of crosslinked polymer.

4. The heat shrink tubing of claim 1, comprising two or more PEBA resins.

5. The heat shrink tubing of claim 1, comprising no fluorinated polymer resin.

6. The heat shrink tubing of claim 1, further comprising one or more additives.

7. The heat shrink tubing of claim 1, wherein the heat shrink tubing has a Tear Index greater than about 0.1.

8. The heat shrink tubing of claim 1, wherein the heat shrink tubing has a Tear Index greater than about 0.5.

9. The heat shrink tubing of claim 1, wherein the heat shrink tubing has a Tear Index greater than about 0.9.

10. The heat shrink tubing of claim 1, wherein the heat shrink tubing has a Tear Index greater than about 0.95.

11. The heat shrink tubing of claim 8, wherein the heat shrink tubing has a recovery ratio (RR) greater than about 1.05:1.

12. The heat shrink tubing of claim 8, wherein the heat shrink tubing has a recovery ratio (RR) greater than about 1.3:1.

13. The heat shrink tubing of claim 8, wherein the heat shrink tubing has a recovery ratio (RR) greater than about 1.6:1.

14. The heat shrink tubing of claim 8, wherein the heat shrink tubing has a recovery ratio (RR) greater than about 2.0:1.

15. The heat shrink tubing of claim 2, wherein the heat shrink tubing has a recovery ratio (RR) greater than about 2.0:1.

16. The heat shrink tubing of claim 15, wherein the heat shrink tubing has a Tear Index greater than about 0.5.

17. The heat shrink tubing of claim 3, wherein the heat shrink tubing has a recovery ratio (RR) greater than about 2.0:1.

18. The heat shrink tubing of claim 17, wherein the heat shrink tubing has a Tear Index greater than about 0.5.

19. A medical device comprising the heat shrink tubing of claim 1.

20. The medical device of claim 19, wherein the medical device is a catheter.
